(19) United States
(12) Patent Application Publication
(10) Pub. No.: US 2025/0161677 A1
(43) Pub. Date: May 22, 2025

(54) MUSCLE STIMULATION VIA BRAIN WAVE SIGNALS TRANSMITTED THROUGH BODY COMMUNICATION

(71) Applicant: Microchip Technology Incorporated, Chandler, AZ (US)

(72) Inventor: Valentin Stoia, Bucharest (RO)

(73) Assignee: Microchip Technology Incorporated, Chandler, AZ (US)

(21) Appl. No.: 18/510,714

(22) Filed: Nov. 16, 2023

(51) Int. Cl. A61N 1/36; H04B 13/00

(52) U.S. Cl. CPC A61N 1/36031; A61N 1/36003; H04B 13/005

(57) ABSTRACT

Systems and methods for communicating brain waves or control signals via body communication from the brain to body extremities to control or activate body parts or even external devices. An EEG coupler/transceiver couples to a person's scalp, wherein the EEG coupler/transceiver comprises an EEG electrode to receive a brain wave from the person, an EEG body communication coupler and an EEG antenna to transmit a signal via the EEG body communication coupler. An activator coupler/transceiver couples to the person's body to stimulate a muscle of the person's body, wherein the activator coupler/transceiver comprises a muscle activator, an activator body communication coupler, and an activator antenna to receive the signal via the activator body communication coupler.

### PRIORITY

This application claims priority to U.S. Provisional Patent Application No. 63/457,501, filed Apr. 6, 2023, the contents of which are hereby incorporated in their entirety.

### TECHNICAL FIELD

The present disclosure relates to an electroencephalography (EEG) based control of paralyzed body muscle(s), in particular, EEG/actuator systems and methods that communicate brain wave control signals to body extremities through the body itself.

### BACKGROUND

Many people have paralyzed body parts due to various reasons. Paralyzed persons cannot control certain parts of their bodies (arms and legs). EEG headsets are used to receive brain wave control signals that are transmitted via wire harnesses to remote actuators used to control or stimulate muscles in body extremities.

Conventional EEG headsets are hard to wear and look unpleasant due to the high number of wires extending from each of the EEG sensors attached at the person's scalp. Conventional wireless EEG electrodes use pure radio frequency signals (Bluetooth, WiFi, Zigbee, etc.) to broadcast data to the entire surrounding environment, therefore being subject to issues like noise, higher power consumption and even undesired data breaches.

One solution proposed by Neuralink Corp, of Fremont, California, is to implant a brain-computer interface device under the scalp, which exposes the patient to health risks and may be regarded as “too dangerous” by patients. The implanted device may transmit neural signals wirelessly to a handheld computer device, like a smartphone, which decodes the data stream into control signals for actions and intents.

There is a need for EEG/actuator systems and methods that communicate brain wave control signals to remote actuators used to control or stimulate muscles in body extremities without wires.

### SUMMARY OF THE INVENTION

Aspects provide an EEG-based control of paralyzed body part(s) through a body communication interface. Aspects of EEG/actuator systems and methods communicate brain wave control signals to remote actuators used to control or stimulate muscles in body extremities via body communication without wires.

An aspect provides a method comprising: coupling a first EEG coupler/transceiver to a person's scalp, wherein the first EEG coupler/transceiver comprises a first EEG electrode and a first EEG body communication coupler; coupling a first activator coupler/transceiver to the person's body, wherein the first activator coupler/transceiver comprises a first muscle activator and a first activator body communication coupler; receiving a first brain wave from the person's brain by the first EEG coupler/transceiver; transmitting a first signal, based on the received first brain wave, from the first EEG body communication coupler of the first EEG coupler/transceiver to the first activator body communication coupler of the first activator coupler/transceiver through the person's body; and stimulating a first muscle of the person's body with the first muscle activator of the first activator coupler/transceiver at least partially based on the first signal.

According to an aspect, there is provided a system comprising: a first EEG coupler/transceiver to couple to a person's scalp, wherein the first EEG coupler/transceiver comprises a first EEG electrode to receive a first brain wave from the person, a first EEG body communication coupler, and a first EEG antenna to transmit a first signal via the first EEG body communication coupler based on the received first brain wave; and a first activator coupler/transceiver to couple to the person's body and to stimulate a first muscle of the person's body, wherein the first activator coupler/transceiver comprises a first muscle activator, a first activator body communication coupler, and a first activator antenna to receive the first signal via the first activator body communication coupler.

An aspect provides system comprising: a first EEG coupler/transceiver to receive a first brain wave from a person and to transmit a first signal, based on the received first brain wave, as a body communication radio frequency (RF) signal; and a first activator coupler/transceiver to receive the first signal and to stimulate a first muscle of the person's body based on the received first signal.

### BRIEF DESCRIPTION OF THE DRAWINGS

The figures illustrate aspects of systems and methods using body communication to communicate brain wave signals to control muscles, wherein systems have two components: an EEG coupler/transceiver, which may include a set of EEG electrodes equivalent to an EEG headset that generates control signals based on brain activity; and an activator coupler/transceiver that causes something to happen in response to control signals from the set of EEG electrodes.

FIG. 1A shows how a body communication radio frequency (RF) signal transmission uses capacitive coupling between the human body, and an EEG coupler/transceiver placed on a person's head, and an activator coupler/transceiver placed on the person's body, for example a leg.

FIG. 1B shows a set of EEG coupler/transceivers on a person's head and a block diagram of an EEG coupler/transceiver.

FIG. 1C shows an activator coupler/transceiver on a person's leg and a block diagram of the activator coupler/transceiver.

FIG. 2 shows a block diagram of a body communication transmitter of an EEG coupler/transceiver.

FIG. 3 shows a multiplex transmission schedule for a set of EEG coupler/transceivers to broadcast signals via body communication.

FIG. 4 shows a schematic diagram of a coordinator EEG coupler/transceiver, a set of EEG coupler/transceivers, and an activator coupler/transceiver.

FIG. 5 shows a flow chart of a method for using body communication of brain wave signals to control body muscles.

FIG. 6 shows a block diagram of circuits for body communication of brain wave signals to control body muscles.

FIG. 7 shows a block diagram of circuitry that may be used to implement various functions, operations, acts, processes, and/or methods for body communication of brain wave signals to control body muscles.

The reference number for any illustrated element that appears in multiple different figures has the same meaning across the multiple figures, and the mention or discussion herein of any illustrated element in the context of any particular figure also applies to each other figure, if any, in which that same illustrated element is shown.

### DESCRIPTION

Aspects provide EEG/actuator systems and methods that communicate brain waves or control signals via body communication to body extremities without wires and without being subject to environmental signal interference. Aspects translate brain waves into control signals and transmit them via body communication to control or activate body parts or even external devices, such as electrical muscle stimulators, through an approach that allows for scalability and upgrading. As used herein, a person's body includes all parts of the body including without limitation: the head, neck, torso, arms, legs, hands, and feet. A body communication includes, without limitation, communication through any part of a person's body, for examples, without limitation: (1) through the head; (2) through the head and neck; (3) through the head, neck, and arm; (4) through the head, neck, arm, and hand; (5) through the head, neck, and torso; (6) through the head, neck, torso, and leg; (7) through the head, neck, torso, leg, and foot; (8) through the torso; (9) through an arm, the torso, and a leg.

According to an aspect, there is provided a system that has two components: an EEG coupler/transceiver, which may include a set of EEG electrodes equivalent to electrodes of an EEG headset that generates control signals based on brain activity; and an activator coupler/transceiver that causes something to happen in response to control signals from the EEG coupler/transceiver. For example, the activator coupler/transceiver may be an electro muscle stimulation device for stimulating a paralyzed leg or other limb.

The EEG coupler/transceiver may provide the functionality of a conventional EEG headset, however, in this case there are no wires inserted into the user brain, or connecting the EEG coupler/transceiver to the activator coupler/transceiver and there is also no broadcast wireless communication beyond the patient user. In this aspect, the EEG coupler/transceiver comprises a set of standalone, self-powered EEG communication nodes, wherein respective nodes have an EEG electrode that is attached to the head skin or scalp of the user to receive an EEG brain signal of the patient user, but it has no wired connection with anything else around it to communicate the EEG brain signal. Rather, respective EEG communication nodes of the EEG coupler/transceiver—besides monitoring the brain wave reading through an analog-to-digital (ADC) channel—is also fitted with a body communication interface (including a small antenna). Via the body communication interface, the EEG communication node communicates the EEG brain signal. Thus, respective EEG communication nodes comprise an EEG electrode and a body communication interface. Additionally, respective EEG communication nodes may have its own power supply (may also be photovoltaic), microcontroller (MCU) with ADC channel, and body communication antenna.

Analogue to digital conversion may form part of the EEG coupler/transceiver to allow signals to be represented in the digital domain and processed by an MCU. For this wearable, battery powered application, power consumption may be a design parameter. An analogue EEG brain signal may be converted to digital ADC data.

A BODYCOM™ system employs a user's body and/or RF conductive materials as a medium for RF signal transmission. The BODYCOM™ body communication system is a short-range wireless connectivity technology that uses the radio frequency (RF) transmission capability of the human body, or other materials having RF transmission capabilities, to transport radio frequency signals that provide safe communications between two or more electronically compatible wireless devices. Communication between BODYCOM™ system devices may occur when they are proximate to, e.g. within a few centimeters of, a human body: simple proximity or touch detection may establish a BODYCOM™ system connection BODYCOM™ technology is more fully described in Microchip Technology Incorporated Application Note AN1391 “Introduction to the BodyCom Technology,” (2011), available at www microchip.com, and its content in its entirety is hereby incorporated by reference herein for all purposes. BODYCOM™ is a trademark owned by Microchip Technology Incorporated, a corporation registered in the state of Delaware, and having an address of 2355 West Chandler Boulevard, Chandler, Ariz. 85224-6199 BODYCOM™ body communication systems are also disclosed in U S. Patent Publication 2015/0044969, wherein its content in its entirety is hereby incorporated by reference herein for all purposes.

Referring now to the drawings, the details of example embodiments are schematically illustrated. Like elements in the drawings are represented by like numbers, and similar elements are represented by like numbers with a different lower case letter suffix.

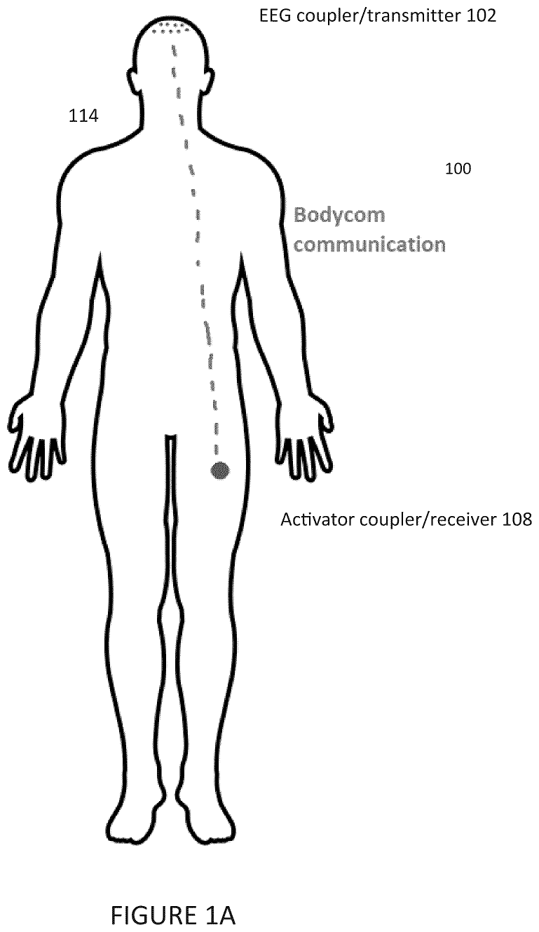

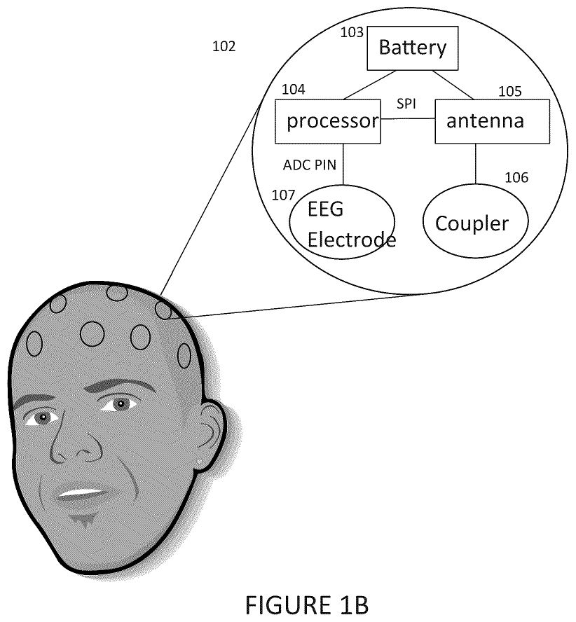

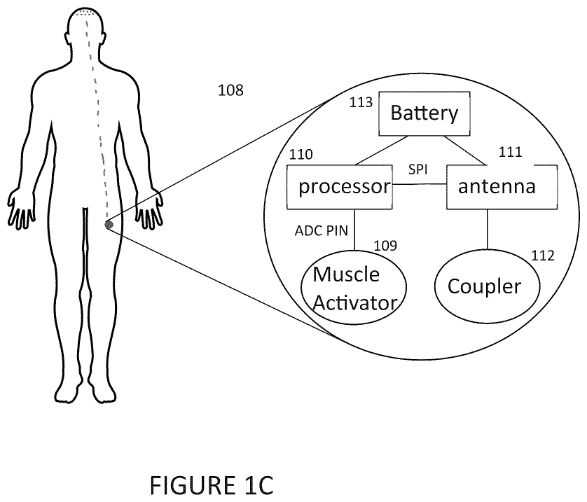

Referring to FIGS. 1A-1C, a schematic block diagram of a system 100 is shown. The system 100 may provide muscle activation via brain wave signals transmitted through body communication, and may comprise an EEG coupler/transceiver 102 and an activator coupler/transceiver 108. The BEG coupler/transceiver 102 may comprise an EEG battery 103, an EEG processor 104, an EEG electrode 107, an EEG antenna 105, and an EEG body communication coupler 106 connected thereto. The activator coupler/transceiver 108 may comprise an activator battery 113, an activator processor 110, a muscle activator 109, an activator antenna 111, and an activator body communication coupler 112 connected thereto. When the EEG body communication coupler 106 and activator body communication coupler 112 are respectively attached to, or brought within proximity of, the body 114 of a person (user), a body communication link may be established between the EEG coupler/transceiver 102 and the activator coupler/transceiver 108. Data may be capacitively transmitted between the EEG body communication coupler 106 and the activator body communication coupler 112 through the user's body 114 using, for example but not limited to, a low-frequency Amplitude Shift Key (ASK) format.

Due to the high permittivity of the human body 114 at low frequencies, transmissions may be achieved with a body communication system operating from about 60 kHz to about 30 MHz. Because data between the EEG coupler/transceiver 102 and the activator coupler/transceiver 108 is communicated through the body 114 of a user, there are substantially no RF transmission detection security issues. The body communication system may provide: 1) very low power consumption, especially for the activator coupler/transceiver 108, 2) fast system response time, 3) stable and robust communication with fault detection, and 4) low cost and complexity of EEG coupler/transceiver 102 and activator coupler/transceiver 108.

The body communication RF signal transmission uses capacitive coupling between the human body 114, the EEG coupler/transceiver 102 and the activator coupler/transceiver 108 as shown in FIG. 1A. Because of this, the RF signals are more attenuated at low frequencies than at high frequencies, wherein the signals that are transmitted for the lower frequencies may be provided of higher amplitude. Both the EEG coupler/transceiver 102 and the activator coupler/transceiver 108 may be battery-powered devices.

Another constraint in frequency choice is the result of the activator processor's 110 power consumption in receive mode. Low-power receiver circuits may enable a receiving frequency in the 60-400 KHz range A frequency between about 6 MHz and about 13 MHz may be used for the transmitting channel, wherein the relation between the power consumption and the performance may be optimum within that frequency range. A frequency of about 128 kHz may be chosen for the channel transmitted from the EEG processor 104 and received by the activator processor 110. A frequency of about 8 MHz may be used for the channel transmitted from the activator processor 110 and received by the EEG processor 104, wherein frequency may be implemented with an on-chip, programmable oscillator of an integrated circuit microcontroller.

For EEG-to-Activator signal transmission in system 100, the communication may be initiated by the EEG processor 104. To start a communication, the system 100 is coupled with the human body 114, and a RF transmission may be transmitted from the EEG coupler/transceiver 102 to search for an activator coupler/transceiver 108. This generated sequence may be applied through a digital driver in the BEG processor 104 to an inductor-capacitor (LC) circuit (not shown) working in a resonant mode. This LC circuit (not shown) may be connected to a pad of the EEG body communication coupler 106 that then transfers the low frequency RF signal to the human body 114. In this way, the human body 114 becomes an extension of the EEG body communication coupler 106, allowing the transfer of the low frequency RF signal to the activator body communication coupler 112 of the activator coupler/transceiver 108 that may be in proximity with the human body 114. A touch is considered herein to be when the human body 114 and a coupling element are in contact or close proximity to one another. The body communication couplers 106 and 112 may be in actual physical contact with the body 114, or they may be close, wherein there may be intermediate layers of clothing, shoe leather, gloves, without limitation, located between the human body 114 and a body communication coupler 106 and 112.

Where the EEG coupler/transceiver 102 may deliver more power to its transmitting channel, a low frequency, e.g., 128 kHz, may be used for the EEG coupler/transceiver 102 transmitting channel. The EEG coupler/transceiver 102, being a fixed part of the system, may be powered externally or can have a bigger battery attached thereto. The signal generated by the EEG coupler/transceiver 102 may be transmitted to the activator coupler/transceiver 108 via the body 114, which is typically waiting in a signal receive mode (low-power consumption). The signal transmitted from the EEG coupler/transceiver 102 may be received by the activator processor 110 and may cause a wake-up of the activator coupler/transceiver 108.

After the signal transmitted from the EEG processor 104 is received by the activator processor 110, it decodes the data and, if a response is necessary, will respond. The activator processor 110 may also be capacitively coupled with the human body 114 through the associated activator body communication coupler 112. It may be preferable that the activator processor 110 use the lowest power consumption in its transmission mode and have more efficient signal coupling, therefore transmitting at high frequencies may be preferred High frequency signals are readily available from a microcontroller and may be used to generate, for example but not limited to, a transmitting signal at about 8 Mhz. The response is sent by the activator processor 110 operating at a high frequency, e.g., about 8 MHz; the signal is carried by the human body 114 to the vicinity of the EEG coupler/transceiver 102 and may be applied through the EEG body communication coupler 106 to the EEG processor 104. See FIG. 1B.

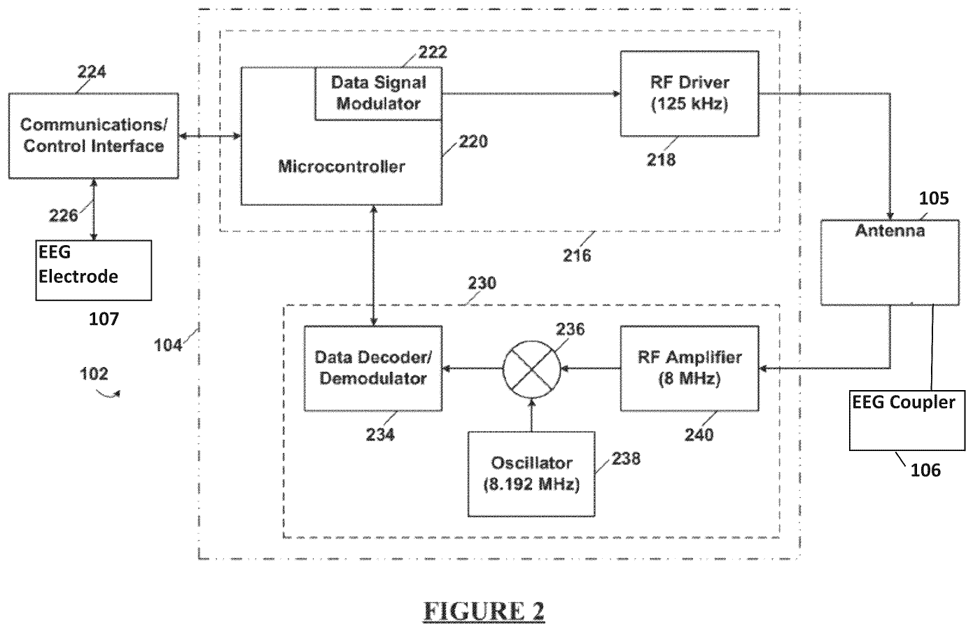

Referring to FIG. 2, depicted is a block diagram of an EEG coupler/transceiver according to one aspect. The EEG coupler/transceiver, generally represented by the numeral 102 may comprise a low frequency transmitter 216, a high frequency receiver 230, an EEG body communication coupler 106 or touch pad in communication with an antenna 105, and a communications and control interface 224 that may have an Ethernet and/or serial communications port 226 for communication with EEG electrode 107. The low frequency transmitter 216 may comprise a data signal modulator 222, and an RF driver 218, which RF driver may be in communication with antenna 105. The data signal modulator 222 may be provided in a microcontroller 220. The high frequency receiver 230 may comprise an RF amplifier 240, a frequency translation mixer 236, an oscillator 238, signal filtering (not shown), and a data decoder/demodulator 234. The demodulated output from the data decoder/demodulator 234 may be coupled to the microcontroller 220, and can receive a complete data sequence and decode incoming data transmitted from the activator coupler/transceiver 108.

The EEG coupler/transceiver 102 may be adapted to perform the following functions: (1) send a challenge to the activator coupler/transceiver 108, (2) receive and decode incoming data from activator coupler/transceiver 108, and (3) provide a communication/control interface 224 for integration with EEG electrode 107 via the communications port 226. The low frequency transmitter 216 and the high frequency receiver 230 may be managed (controlled) by a microcontroller 220 of the processor 104, wherein the processor 104 can manage the transmission/receiving process, perform encoding/decoding and error detection. In addition, the processor 104 may support implementation of a security algorithm for secure communication. The processor 104 may receive brain wave signals via the BEG electrode 107 and generate a control signal based on the brain wave signals. The transmitter 230 may drive the EEG antenna 107 that may further support the BEG body communication coupler 106 (see Microchip Application Note AN1391, touch pad).

The placement and individual role of each EEG coupler/transceiver 102 follows the settings of conventional EEG readers or headsets. For example, the EEG coupler/transceiver system may be a 1, 2, 24, 32, or 64-node system having as many EEG coupler/transceivers 102.

As shown in FIG. 4, a set of EEG coupler/transceivers 102 may use a coordinator EEG coupler/transceiver 101 to read the EEG data pattern ADC data from the other EEG coupler/transceivers 102 (via body communication), and then the coordinator EEG coupler/transceiver 101 may output the EEG data pattern ADC data to an activator coupler/transceiver 108. Alternatively, the coordinator EEG coupler/transceiver may generate a control signal from the collected the EEG data pattern ADC data, and transmit the control signal to an activator coupler/transceiver 108.

One EEG coupler/transceiver of a set, called “NO” for reference, may be a coordinator EEG coupler/transceiver 101 because it also maintains communication scheduling. Coordinator EEG coupler/transceiver 101 can occasionally do a scheduling broadcast, which will be received by the other EEG coupler/transceivers 102 (called N1, N2, N3 . . . . Nn for reference) to know the time each of them is to transmit the respective EEG data pattern ADC data via body communication without the risk of having a data traffic collision. Therefore, most, if not all, EEG coupler/transceivers 102 can perform both-way data exchange (send and receive) via their body communication interfaces (antenna and couplers).

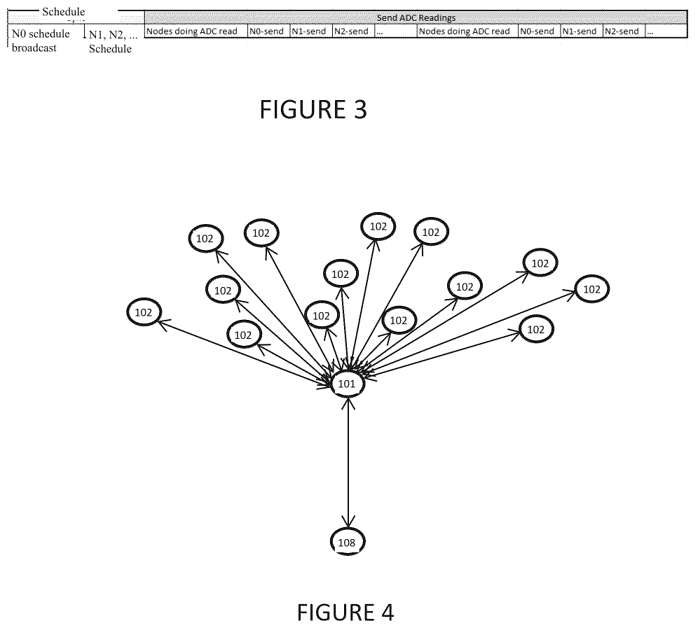

As shown in FIG. 3, a table shows reference timing: At a first time period, Coordinator EEG coupler/transceiver 101 may send an NO scheduling broadcast - - - at a second time period EEG coupler/transceivers 102 N1, N2, may receive the NO scheduling broadcast and wait to transmit until scheduled transmissions . . . . At a 3^{rd} time period nodes NO to Nn may perform an ADC read - - - at a 4^{th} time period Coordinator EEG coupler/transceiver 101 N0 may transmit its ADC data - - - at a 5^{th} time period EEG coupler/transceiver 102 N1 may transmit its ADC data - - - at a 6^{th} time period EEG coupler/transceiver 102 N2 may transmit its ADC data. The cycle may then be repeated in accordance with the original schedule. In one aspect, as each EEG coupler/transceiver 102 may start the data string with its specific digital ID, the activator coupler/transceiver 108 reads all the EEG ADC data from all the EEG coupler/transceivers 102 and decides if the full EEG data pattern matches the profile of the activator coupler/transceiver 108. The EEG data pattern may not be directed to the activator coupler/transceiver 108, and may be directed to another activator coupler/transceiver 108. If the activator coupler/transceiver 108 determines the full EEG data pattern matches its profile, the activator coupler/transceiver 108 may generate a control signal to engage its muscle activator 109. Also, it may take several readings for a valid instruction to be identified from the EEG data pattern, and in some cases the EEG data pattern is inconclusive.

FIG. 4 shows a system for transmitting body communication signals between a coordinator EEG coupler/transceiver 101 and an activator coupler/transceiver 108. The system also has a plurality of EEG coupler/transceivers 102 that each receive a brain wave from a person's brain and transmit a body communication signal to the coordinator EEG coupler/transceiver 101. The coordinator EEG coupler/transceiver 101 schedules when each EEG coupler/transceivers 102 may serially transmit a body communication signal to avoid traffic collisions. The coordinator EEG coupler/transceiver 101 may multiplex transmission of body communication signals. In one aspect, the coordinator EEG coupler/transceiver 101 also transmits all of the body communications from the EEG coupler/transceivers 102 to the activator coupler/transceiver 108, and the activator coupler/transceiver 108 determines whether to stimulate a muscle of the body based on the body communications. In another aspect, the coordinator EEG coupler/transceiver 101 generates a control body communication signal based on the body communication signals received from the EEG coupler/transceivers 102, transmits the control body communication signal to the activator coupler/transceiver 108, and the activator coupler/transceiver 108 stimulates a muscle of the body based on the control body communication signal.

An EEG coupler/transceiver 102 or an activator coupler/transceiver 108 may output an EEG data pattern to a remote device via a cable or wirelessly (Bluetooth, WiFi, UWB, without limitation). According to one aspect, a coordinator EEG coupler/transceiver 101 may transmit an EEG data pattern to an activator coupler/transceiver 108 via a cable or wirelessly (Bluetooth, WiFi, UWB, without limitation), so that body communication is only used by the EEG coupler/transceivers 102 of the EEG “headset,” with the coordinator EEG coupler/transceiver 101 which may increase its interoperability with other devices.

The muscle activator 109 (see FIG. 1C) of the activator coupler/transceiver 108 may be an electro muscle stimulation device similar to a conventional functional electrical stimulation (FES) device. The activator coupler/transceiver 108 may also be fitted with an activator coupler 112 and an activator antenna 111 to receive input from the EEG coupler/transceiver 102 via body communications.

Alternatively, the system 100 may comprise a plurality or set of activator coupler/transceivers 108, wherein respective activator coupler/transceivers 108 comprises a muscle activator 109 and a body communication interface comprising an activator body communication coupler 112 and an activator antenna 111. Additionally, respective activator coupler/transceivers 108 may have its own power supply such as a battery 113 (may also be photovoltaic), and a processor 110 such as an MCU. The activator body communication antenna 111 can be placed on the body a distance from the muscle activator 109, so that the locally applied muscle stimulation voltage (30V, for example) does not overlap with the body communication data influx.

Electroencephalography (EEG) electrodes 107 may be sensors that read brain waves (electromagnetic fluctuations generated by brain activity) from which control signals may be generated to enable control of body parts or even external devices. The EEG coupler/transceiver 102 may be wireless, utilizing body communication, which provides a more acceptable visual impression for patients and provides more portability. Because EEG coupler/transceivers 102 do not broadcast EEG data to the surrounding environment, issues with noise, high power consumption, and data breaches may be avoided. Aspects may be less susceptible to RF noise or data hacking. Debugging which of the EEG coupler/transceivers 102 in an EEG headset is not functioning properly can be easily done by bringing a body communication data sniffer into proximity of the body. On the other hand, data sent by the EEG coupler/transceivers 102 of an EEG headset can also be encrypted, in case data protection is also desired. “Raw EEG data” broadcast to a person's entire body means that any muscle stimulation device can be removed or added anywhere on the body, at any given time.

Potential industrial applications of aspects include: medical (for example, control of paralyzed limbs to assist a paralyzed person to regain muscle control); entertainment (for example, virtual reality gaming); industrial (for example, controlling mechanical arms); and military (for example, to control bionic hardware or other electromechanical parts attached to the human body).

Due to the high permittivity of the human body at low radio frequencies, body communications can be achieved via radio frequency transmission and reception devices operating at any frequency(ies) from about 60 kHz to about 30 MHz. Furthermore, a battery powered device, e.g., cell phone, smart phone, personal digital assistant, game tablet, touch pad computer, portable computer, without limitation, can stay in a very low power standby or sleep state. Then capacitive proximity sensing may be used to wake-up the device when touched or in close proximity thereto for further processing of the radio frequency signals received through the closely coupled part of the user's body 114.

A short-range wireless signal may be transmitted over “skin” of the user's body 114 with a very short proximity range in air. Thus two devices, both in close proximity with a person (user), e.g., through clothing, gloves, near to or touching skin, without limitation, can communicate, but a device not in close proximity to the person (user) may not be able to communicate.

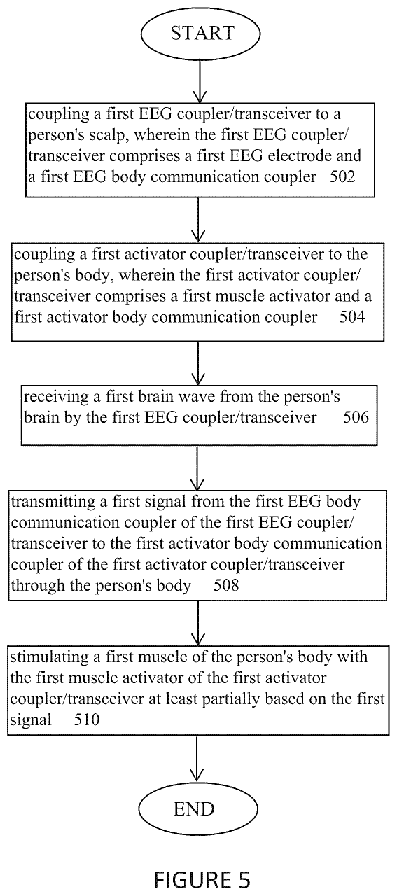

FIG. 5 shows a flow chart for a method of communicating brain waves or control signals via body communication to body extremities to control or activate body parts or even external devices. A first EEG coupler/transceiver is coupled 502 to a person's scalp, wherein the first EEG coupler/transceiver comprises a first EEG electrode and a first EEG body communication coupler. A first activator coupler/transceiver is coupled 504 to the person's body, wherein the first activator coupler/transceiver comprises a first muscle activator and a first activator body communication coupler. A first brain wave is received 506 from the person's brain by the first EEG coupler/transceiver. A first signal, based on the received first brain wave, is transmitted 508 from the first EEG body communication coupler of the first EEG coupler/transceiver to the first activator body communication coupler of the first activator coupler/transceiver through the person's body. The signal may be a representation of the brain wave, or a control signal based on the received brain wave. A first muscle of the person's body is stimulated 510 with the first muscle activator of the first activator coupler/transceiver at least partially based on the first signal.

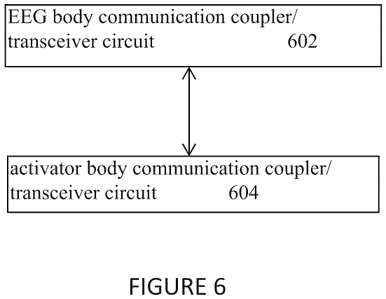

FIG. 6 shows a block diagram of circuitry 600 for communicating brain waves or control signals via body communication to body extremities to control or activate body parts or even external devices. An EEG body communication coupler/transceiver circuit 602 may receive a brain wave from a person and transmit a body communication signal as a body communication radio frequency (RF) signal. An activator body communication coupler/transceiver circuit 604 may receive the body communication signal and stimulate a muscle of the person's body.

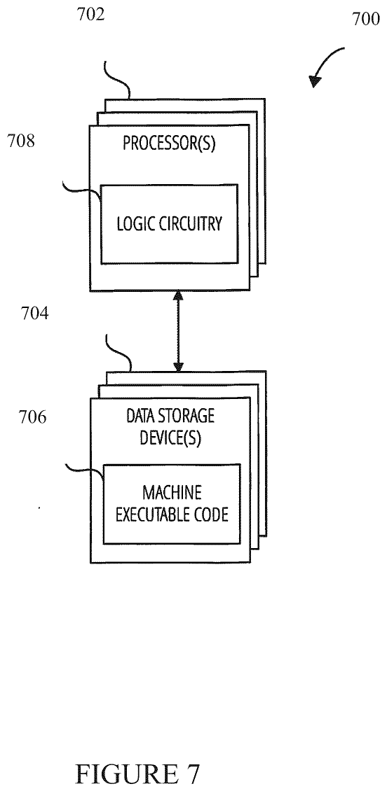

FIG. 7 shows a block diagram of circuitry 700 that may be used to implement various functions, operations, acts, processes, and/or methods. The circuitry 700 includes one or more processors 702 (sometimes referred to herein as “processors 702”) operably coupled to one or more data storage devices (sometimes referred to herein as “storage 704”). The storage 704 includes machine executable code 706 stored thereon and the processors 702 include logic circuitry 708. The machine executable code 706 includes information describing functional elements that may be implemented by (e.g., performed by) the logic circuitry 708. The logic circuitry 708 is adapted to implement (e.g., perform) the functional elements described by the machine executable code 706. The circuitry 700, when executing the functional elements described by the machine executable code 706, should be considered as special purpose hardware configured for carrying out functional elements disclosed herein. In some embodiments the processors 702 may perform the functional elements described by the machine executable code 706 sequentially, concurrently (e.g., on one or more different hardware platforms), or in one or more parallel process streams.

When implemented by logic circuitry 708 of the processors 702, the machine executable code 706 adapts the processors 702 to perform operations of embodiments disclosed herein. For example, the machine executable code 706 may adapt the processors 702 to perform at least a portion or a totality of the method of FIG. 5. As another example, the machine executable code 706 may adapt the processors 702 to perform at least a portion or a totality of the operations discussed for the EEG coupler/transceiver 102, the coordinator EEG coupler/transceiver 101, and the activator coupler/transceiver 108 of FIGS. 1-2 and 4.

The processors 702 may include a general purpose processor, a special purpose processor, a central processing unit (CPU), a microcontroller, a programmable logic controller (PLC), a digital signal processor (DSP), an application specific integrated circuit (ASIC), a field-programmable gate array (FPGA) or other programmable logic device, discrete gate or transistor logic, discrete hardware components, other programmable device, or any combination thereof designed to perform the functions disclosed herein. A general-purpose computer including a processor is considered a special-purpose computer while the general-purpose computer is configured to execute functional elements corresponding to the machine executable code 706 (e.g., software code, firmware code, hardware descriptions) related to embodiments of the present disclosure. It is noted that a general-purpose processor (may also be referred to herein as a host processor or simply a host) may be a microprocessor, but in the alternative, the processors 702 may include any conventional processor, controller, microcontroller, or state machine. The processors 702 may also be implemented as a combination of computing devices, such as a combination of a DSP and a microprocessor, a plurality of microprocessors, one or more microprocessors in conjunction with a DSP core, or any other such configuration.

In some embodiments the storage 704 includes volatile data storage (e.g., random-access memory (RAM)), non-volatile data storage (e.g., Flash memory, a hard disc drive, a solid state drive, erasable programmable read-only memory (EPROM), etc.). In some embodiments the processors 702 and the storage 704 may be implemented into a single device (e.g., a semiconductor device product, a system on chip (SOC), etc.). In some embodiments the processors 702 and the storage 704 may be implemented into separate devices.

In some embodiments the machine executable code 706 may include computer-readable instructions (e.g., software code, firmware code). By way of non-limiting example, the computer-readable instructions may be stored by the storage 704, accessed directly by the processors 702, and executed by the processors 702 using at least the logic circuitry 708. Also by way of non-limiting example, the computer-readable instructions may be stored on the storage 704, transferred to a memory device (not shown) for execution, and executed by the processors 702 using at least the logic circuitry 708. Accordingly, in some embodiments the logic circuitry 708 includes electrically configurable logic circuitry 608.

In some embodiments the machine executable code 706 may describe hardware (e.g., circuitry) to be implemented in the logic circuitry 708 to perform the functional elements. This hardware may be described at any of a variety of levels of abstraction, from low-level transistor layouts to high-level description languages. At a high-level of abstraction, a hardware description language (HDL) such as an IEEE Standard hardware description language (HDL) may be used. By way of non-limiting examples, VERILOG™, SYSTEMVERILOG™ or very large scale integration (VLSI) hardware description language (VHDL™) may be used.

HDL descriptions may be converted into descriptions at any of numerous other levels of abstraction as desired. As a non-limiting example, a high-level description can be converted to a logic-level description such as a register-transfer language (RTL), a gate-level (GL) description, a layout-level description, or a mask-level description. As a non-limiting example, micro-operations to be performed by hardware logic circuits (e.g., gates, flip-flops, registers, without limitation) of the logic circuitry 708 may be described in a RTL and then converted by a synthesis tool into a GL description, and the GL description may be converted by a placement and routing tool into a layout-level description that corresponds to a physical layout of an integrated circuit of a programmable logic device, discrete gate or transistor logic, discrete hardware components, or combinations thereof. Accordingly, in some embodiments the machine executable code 706 may include an HDL, an RTL, a GL description, a mask level description, other hardware description, or any combination thereof.

In embodiments where the machine executable code 706 includes a hardware description (at any level of abstraction), a system (not shown, but including the storage 704) may be configured to implement the hardware description described by the machine executable code 706. By way of non-limiting example, the processors 702 may include a programmable logic device (e.g., an FPGA or a PLC) and the logic circuitry 708 may be electrically controlled to implement circuitry corresponding to the hardware description into the logic circuitry 708. Also by way of non-limiting example, the logic circuitry 708 may include hard-wired logic manufactured by a manufacturing system (not shown, but including the storage 704) according to the hardware description of the machine executable code 706.

Regardless of whether the machine executable code 706 includes computer-readable instructions or a hardware description, the logic circuitry 708 is adapted to perform the functional elements described by the machine executable code 706 when implementing the functional elements of the machine executable code 706. It is noted that although a hardware description may not directly describe functional elements, a hardware description indirectly describes functional elements that the hardware elements described by the hardware description are capable of performing.

Although examples have been described above, other variations and examples may be made from this disclosure without departing from the spirit and scope of these disclosed examples.

1. A method comprising:
coupling a first EEG coupler/transceiver to a person's scalp, wherein the first EEG coupler/transceiver comprises a first EEG electrode and a first EEG body communication coupler;
coupling a first activator coupler/transceiver to the person's body, wherein the first activator coupler/transceiver comprises a first muscle activator and a first activator body communication coupler;
receiving a first brain wave from the person's brain by the first EEG coupler/transceiver;
transmitting a first signal, based on the received first brain wave, from the first EEG body communication coupler of the first EEG coupler/transceiver to the first activator body communication coupler of the first activator coupler/transceiver through the person's body; and
stimulating a first muscle of the person's body with the first muscle activator of the first activator coupler/transceiver at least partially based on the first signal.

2. The method as claimed in claim 1, comprising:
coupling a second EEG coupler/transceiver to a person's scalp, wherein the second EEG coupler/transceiver comprises a second EEG electrode and a second EEG body communication coupler; and
receiving a second brain wave from the person's brain by the second EEG coupler/transceiver.

3. The method as claimed in claim 2, comprising:
coupling a second activator coupler/transceiver to the person's body, wherein the second activator coupler/transceiver comprises a second muscle activator and a second activator body communication coupler;
transmitting a second signal from the second EEG body communication coupler of the second EEG coupler/transceiver to the second activator body communication coupler of the second activator coupler/transceiver through the person's body; and
stimulating a second muscle of the person's body with the second muscle activator of the second activator coupler/transceiver based at least partially on the second signal.

4. The method as claimed in claim 2, comprising:
transmitting a second signal from the second EEG body communication coupler of the second EEG coupler/transceiver to the first activator body communication coupler of the first activator coupler/transceiver through the person's body, based on the received second brain wave; and
stimulating a first muscle of the person's body with the first muscle activator of the first activator coupler/transceiver at least partially based on the first and second signals.

5. The method as claimed in claim 2, comprising:
transmitting, based on the received second brain wave, a second signal from the second EEG body communication coupler of the second EEG coupler/transceiver to the first EEG body communication coupler of the first EEG coupler/transceiver through the person's scalp;
generating, by the first EEG coupler/transceiver, the first signal based at least partially on the first brain wave and the second signal.

6. The method as claimed in claim 2, comprising;
transmitting, based on the second brain wave, a second signal from the second EEG body communication coupler of the second EEG coupler/transceiver; and
scheduling transmitting the first signal and transmitting the second signal.

7. The method as claimed in claim 1, wherein the signal is a signal selected from: the first brain wave, a signal triggered by the first brain wave, and a signal based at least partially on the first brain wave.

8. The method as claimed in claim 1, wherein the first muscle activator of the first activator coupler/transceiver is a functional electrical stimulator.

9. A system comprising:
a first EEG coupler/transceiver to couple to a person's scalp, wherein the first EEG coupler/transceiver comprises a first EEG electrode to receive a first brain wave from the person, a first EEG body communication coupler, and a first EEG antenna to transmit a first signal via the first EEG body communication coupler based on the received first brain wave; and
a first activator coupler/transceiver to couple to the person's body and to stimulate a first muscle of the person's body, wherein the first activator coupler/transceiver comprises a first muscle activator, a first activator body communication coupler, and a first activator antenna to receive the first signal via the first activator body communication coupler.

10. The system as claimed in claim 8, comprising:
a second EEG coupler/transceiver to couple to a person's scalp, wherein the second EEG coupler/transceiver comprises a second EEG electrode to receive a second brain wave from the person, a second EEG body communication coupler, and a second EEG antenna to transmit a second signal via the second EEG coupler based on the received second brain wave.

11. The system as claimed in claim 9, comprising:
a second activator coupler/transceiver to couple to the person's body and to stimulate a second muscle of the person's body, wherein the second activator coupler/transceiver comprises a second muscle activator, a second activator body communication coupler, and a second activator antenna to receive the second signal via the second activator body communication coupler.

12. The system as claimed in claim 9,
wherein the second EEG body communication coupler of the second EEG coupler/transceiver is to transmit the second signal to the first activator body communication coupler of the first activator coupler/transceiver through the person's body; and
wherein the first muscle activator of the first activator coupler/transceiver is to stimulate a first muscle of the person's body based on the first and second signals.

13. The system as claimed in claim 9,
wherein the second EEG body communication coupler of the second EEG coupler/transceiver is to transmit the second signal to the first EEG body communication coupler of the first EEG coupler/transceiver through the person's body, and
wherein the first EEG coupler/transceiver is to generate the first signal based at least in part on the first brain wave and the second signal.

14. The system as claimed in claim 9, wherein the first EEG coupler/transceiver is a coordinator to schedule transmitting body communication signals.

15. The system as claimed in claim 8,
wherein the first signal is a body communication radio frequency (RF) signal, and
wherein the first body communication RF signal is a signal selected from: the first brain wave, a signal triggered by the first brain wave, and a signal based at least partially on the first brain wave.

16. The system as claimed in claim 8, wherein the first muscle activator of the first activator coupler/transceiver is a functional electrical stimulator.

17. A system comprising:
a first EEG coupler/transceiver to receive a first brain wave from a person and to transmit a first signal, based on the received first brain wave, as a body communication radio frequency (RF) signal; and
a first activator coupler/transceiver to receive the first signal and to stimulate a first muscle of the person's body based on the received first signal.

18. The system as claimed in claim 17, comprising:
a second EEG coupler/transceiver to receive a second brain wave from the person and to transmit a second signal as a body communication RF signal based on the received second brain wave; and
a second activator coupler/transceiver to receive the second signal and to stimulate a second muscle of the person's body based on the received second signal.

19. The system as claimed in claim 18, wherein the second EEG coupler/transceiver is to transmit the second signal to the first EEG coupler/transceiver circuit, and wherein the first EEG coupler/transceiver circuit generates the first signal based on the first brain wave and the second signal.

20. The system as claimed in claim 17, wherein the first EEG coupler/transceiver circuit is a coordinator EEG coupler/transceiver circuit to transmit signals according to a schedule.
